(19) United States
(12) Patent Application Publication
(10) Pub. No.: US 2018/0318457 A1
(43) Pub. Date: Nov. 8, 2018

(54) DEVICE FOR DISINFECTING CPAP COMPONENTS AND METHOD OF USING THE SAME

(71) Applicant: 3B Medical, Inc., Winter Haven, FL (US)

(72) Inventor: Albert A. Lucio, Haines City, FL (US)

(21) Appl. No.: 15/956,793

(22) Filed: Apr. 19, 2018

Related U.S. Application Data
(60) Provisional application No. 62/500,648, filed on May 3, 2017.

(51) Int. Cl. A61L 2/00; A61L 2/24; A61M 16/06

(52) U.S. Cl. CPC A61L 2/0047; A61M 16/0066; A61M 16/06; A61L 2/24

(57) ABSTRACT

This disclosure relates to a device for disinfecting CPAP components and a method of using the same. An example device includes a chamber, an ultraviolet (UV) light configured to emit UV light within the chamber, and a control unit configured to activate the UV light for a period of time.

### RELATED APPLICATIONS

This application claims the benefit of U.S. Provisional Application No. 62/500,648, filed May 3, 2017, the entirety of which is herein incorporated by reference.

### TECHNICAL FIELD

This disclosure relates to a device for disinfecting CPAP components and a method of using the same.

### BACKGROUND

Continuous positive airway pressure (CPAP) represents a treatment for patients with breathing problems. Such problems typically manifest themselves at night while the patient is asleep. One such problem is sleep apnea.

The CPAP treatment uses mild air pressure to keep airways open, particularly when a patient is sleeping. CPAP systems have several components. The first is a flow generator, which is essentially a pump that creates a stream of air. Many flow generators include a humidifier, which is typically attached to the flow generator or integrally formed with the same. Humidifiers are configured to heat and moisten the air flow from the flow generator, which reduces the likelihood that a patient will experience discomfort from breathing dry air for a prolonged period. CPAP systems also include a conduit (i.e., a tube or hose) fluidly coupling a mask apparatus to the flow generator. The mask is affixed to the mouth and/or nose of a patient. CPAP systems also include various couplings, fittings, seals, valves, etc., that establish the fluid connection between the flow generator and the patient.

During use over the course of days, weeks, and months, it is recommended that the components of a CPAP system be cleaned and disinfected to prevent buildup of bacteria, for example. Disinfection is recommended to reduce health risks. CPAP systems and their associated components are typically cleaned manually by a patient using soap and water.

### SUMMARY

A device for disinfecting components of a continuous positive airway pressure (CPAP) system according to an exemplary aspect of the present disclosure includes, among other things, a chamber, an ultraviolet (UV) light configured to emit UV light within the chamber, and a control unit configured to activate the UV light for a period of time.

In a further non-limiting embodiment of the foregoing device, the UV light emits UV-C light.

In a further non-limiting embodiment of any of the foregoing devices, the UV light emits UV light at a wavelength of 254 nanometers (nm).

In a further non-limiting embodiment of any of the foregoing devices, the UV light includes a 13 Watt UV-C bulb.

In a further non-limiting embodiment of any of the foregoing devices, the device includes a base and a drawer slidably mounted to the base and moveable relative to the base between an open position and a closed position. The base and drawer provide boundaries of the chamber when the drawer is in the closed position.

In a further non-limiting embodiment of any of the foregoing devices, the control unit is configured such that the UV bulb is only activated when the drawer is in the closed position.

In a further non-limiting embodiment of any of the foregoing devices, the drawer is lined with a reflective material.

In a further non-limiting embodiment of any of the foregoing devices, the reflective material is aluminum.

In a further non-limiting embodiment of any of the foregoing devices, the device includes a base and a lid pivotably mounted to the base and moveable relative to the base between an open position and a closed position. The base and lid provide boundaries of the chamber when the lid is in the closed position.

In a further non-limiting embodiment of any of the foregoing devices, the control unit is configured such that the UV bulb is only activated when the lid is in the closed position.

In a further non-limiting embodiment of any of the foregoing devices, the device includes a heater, and the control unit is configured to activate the heater and the UV light for the period of time.

In a further non-limiting embodiment of any of the foregoing devices, the device includes a blower, and the control unit is configured to activate the blower for the period of time.

In a further non-limiting embodiment of any of the foregoing devices, the period of time is a predefined period of time.

In a further non-limiting embodiment of any of the foregoing devices, the period of time is about 5 minutes.

A method for disinfecting a component of a continuous positive airway pressure (CPAP) system according to an exemplary aspect of the present disclosure includes, among other things, directing ultraviolet (UV) light onto a CPAP component within a chamber for a time period.

In a further non-limiting embodiment of any the foregoing method, the period of time is about 5 minutes.

In a further non-limiting embodiment of any the foregoing method, the UV light is UV-C light.

In a further non-limiting embodiment of any the foregoing method, the UV light is at a wavelength of 254 nanometers (nm).

In a further non-limiting embodiment of any the foregoing method, a boundary of the chamber is provided in part by one of a lid and a drawer, and the UV light is only directed to the CPAP component when the one of the lid and the drawer is closed.

In a further non-limiting embodiment of any the foregoing method, the method includes directing heat to the CPAP component for the period of time, and activating a blower for the period of time.

### BRIEF DESCRIPTION OF THE DRAWINGS

FIG. 1 illustrates an example device for disinfecting CPAP components from a perspective view. Certain aspects of the device are illustrated schematically. In FIG. 1, a lid of the device is in an open position.

FIG. 2 illustrates the device of FIG. 1 with the lid in a closed position.

FIG. 3 illustrates another example device for disinfecting CPAP components from a perspective view. In FIG. 3, the drawer of the device is in a closed position.

FIG. 4 illustrates the device of FIG. 3 with the drawer in the open position.

### DETAILED DESCRIPTION

This disclosure relates to a device for disinfecting CPAP components (or, CPAP equipment), including the various parts of a CPAP system, such as hoses, masks, pillows, couplings, humidifiers, etc., that require frequent cleaning and disinfecting. The disclosed device provides a reliable and easy-to-use chamber that is large enough to hold the most commonly cleaned components of a CPAP machine, such as a mask, hose, and humidifier. A patient can place one or more CPAP components in the chamber, close the chamber, and begin a relatively short disinfecting cycle. An example device includes a chamber, an ultraviolet (UV) light configured to emit UV light within the chamber, and a control unit configured to activate UV light for a period of time. Following the time period, which is relatively short, the components within the device are disinfected at a substantially high disinfection rate.

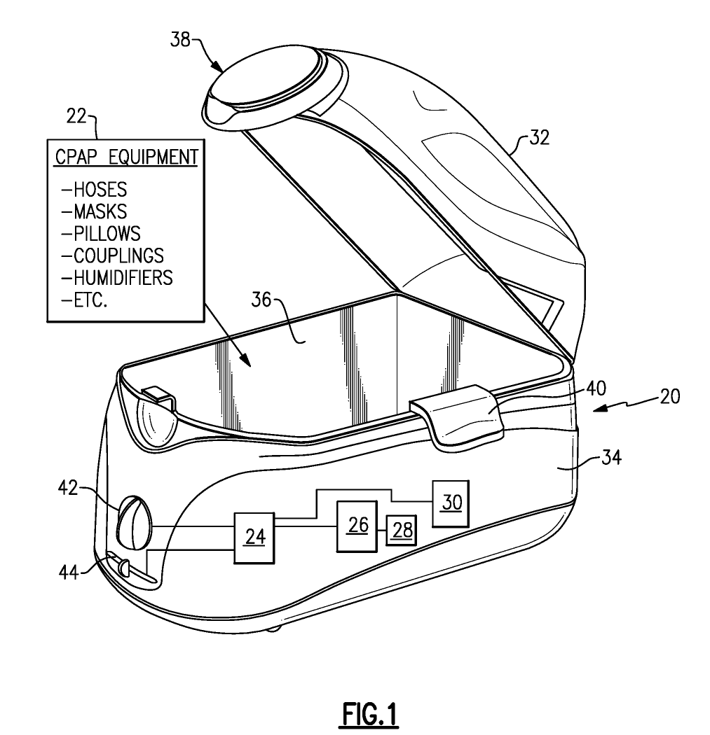

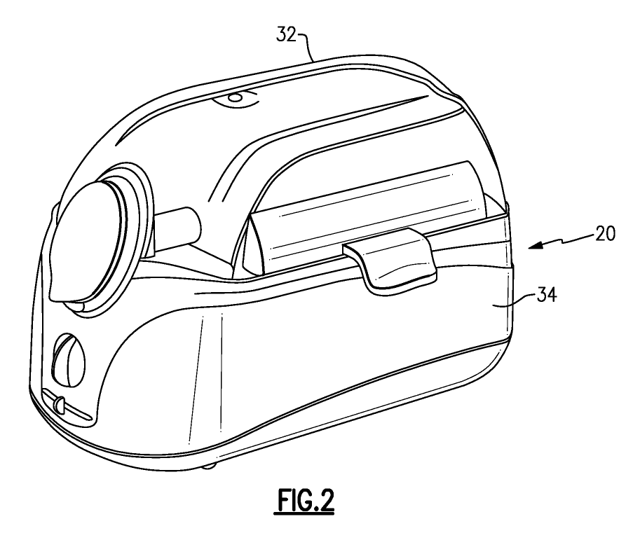

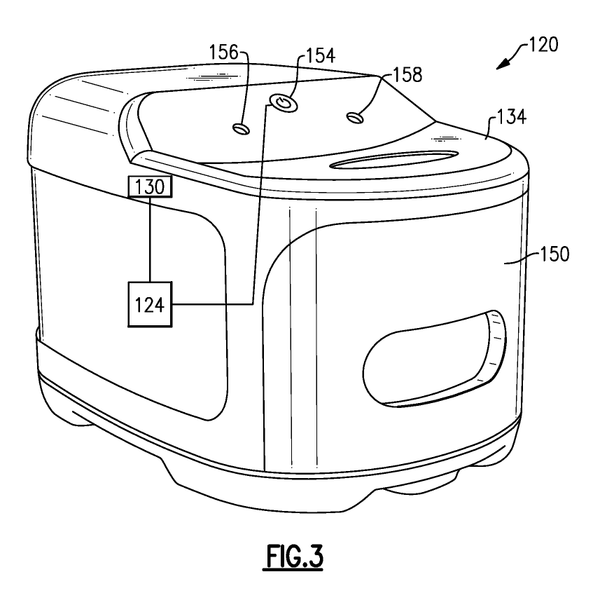

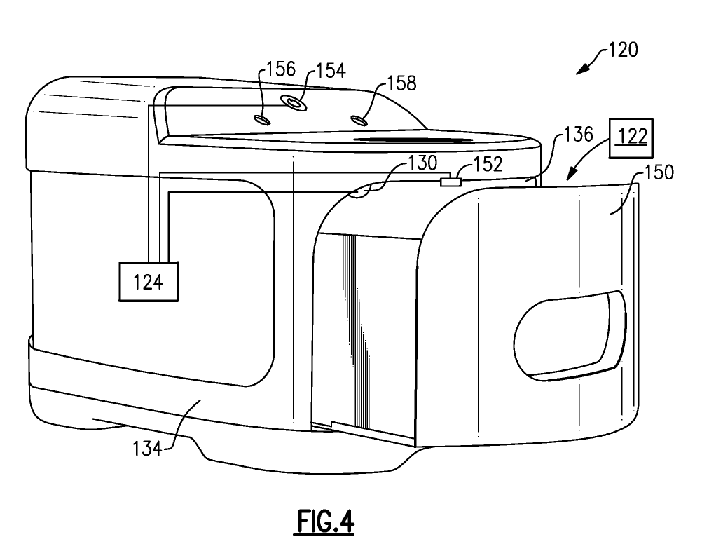

FIGS. 1 and 2 illustrate an example device 20 for disinfecting CPAP components 22 from a perspective view. In the embodiment of FIGS. 1 and 2, the device 20 includes a number of customizable operating parameters, which may be set by the user, as will be discussed below. The device 20 may be particularly useful in laboratory settings, where users are trained to set these parameters. This disclosure also relates to a device, such as that described relative to FIGS. 3 and 4, which does not allow a user to set any operating parameters. The latter device may be more user friendly from the perspective of some users, and may be more applicable for residential applications.

With continued reference to FIGS. 1 and 2, the device 20 includes a control unit 24, heater 26, blower (e.g., fan) 28, and an ultraviolet (UV) light 30. It should be understood that the CPAP components 22, control unit 24, heater 26, blower 28, and UV light 30 are illustrated schematically.

The control unit (sometimes called a “controller”) 24 may be programmed with executable instructions for interfacing with and operating the various components of the device 20, including but not limited to those shown in the figures and discussed herein. It should also be understood that the control unit 24 may additionally include a combination of hardware and software, and specifically may include a processing unit and non-transitory memory for executing the various control strategies and modes of the device 20.

The UV light 30 is selectively activated in response to instructions from the control unit 24. The UV light 30 in this example is provided by a light source, specifically a UV bulb (sometimes called a “UV lamp”), which is configured to emit UV light. The UV light 30 may be provided by a 13 Watt UV-C bulb, in one example. In other examples, the UV light 30 is provided by a bulb within a range of 5 to 20 Watts.

In a particular example, the UV light 30 is configured to emit UV-C light, which is a subtype of UV light especially suited for disinfection. Specifically, UV-C is relatively short-wavelength UV light, which is known to kill or inactivate microorganisms such as bacteria. In one example, the UV light 30 emits UV light at a wavelength within a range of 250 to 270 nanometers (nm), and in one particular example the UV light has a wavelength of 254 nm.

The device 20 includes a lid 32 pivotably mounted to a base 34 and configured to pivot between an open position (FIG. 1) and a closed position (FIG. 2). In the open position, one or more CPAP components 22 can be provided into a chamber 36 within the device. The chamber 36 is partially defined by the lid 32 and partially defined by the base 34. When the lid 32 is closed, the lid 32 and base 34 define an enclosed chamber 36. The control unit 24 is configured to determine when the lid 32 is closed, such as in response to a signal from a sensor, such as a magnetic switch. The lid 32 and base 34 are sized such that the chamber 36 can hold a number of pieces of CPAP components.

The lid 32 is held in place in the closed position by way of a handle 38, which includes a latch, and one or more clasps 40. In this example, there is only one clasp 40 on a side of the base 34, but it should be understood that the device 20 could include additional clasps 40. Further, the lid 32 and 34 are sized and arranged such that, when the lid 32 is in the closed position, a seal is established. During use of the device 20, there may be relatively hot air flowing within the chamber 36. The handle 38 (which includes a latch) and clasp 40 ensures that the lid 32 stays closed and sealed during operation.

In one example, when the lid 32 is in the closed position, the chamber 36 can hold at least a humidifier, hose, and mask. This disclosure is not limited to these CPAP components 22, and can be used to disinfect other pieces of CPAP components 22, including but not limited to hoses, masks, pillows, humidifiers, couplings, fittings, seals, valves, etc. To this end, this disclosure is not even limited to use with CPAP components. For instance, the device 20 can be used to disinfect other medical, dental, and hygiene-related products, such as toothbrushes, hearing aids, dentures, pacifiers, etc.

Operation of the device 20 is regulated by the control unit 24, which is electrically coupled in this example to a first dial 42, a second dial 44, the heater 26, the blower 28, and the UV light 30.

The first dial 42 is a rotatable knob and is configured to control a temperature setting in one example. The control unit 24 is configured to interpret the input from the first dial 42, and regulate operation of the heater 26 (which could be any known type of heater, such as a coil heater) and blower 28 accordingly. The second dial 44 is an adjustable slider and is configured to control a time setting in one example. The control unit 24 is configured to interpret the input from the second dial 44 and operate the heater 26, blower 28, and UV light 30 for the set time. It should be understood that the first and second dials 42, 44 could be different types of input devices, and are not limited to knobs and sliders.

In one example process, a user places CPAP components 22 within the chamber 36 and closes the lid 32. In the example of FIG. 1, the user sets the first dial 42 and second dial 44, and presses an “on” button. Alternatively, there is no “on” button and the process begins automatically when the lid 32 is closed.

The control unit 24 is configured to instruct the heater 26 to heat air within the chamber 36, and the control unit 24 is further configured to activate the blower 28 and UV light 30. The blower 28 distributes heated air through the chamber 36 and evenly heats the CPAP components 22 using convection. During this process, the UV light 30 emits ultraviolet light to kill or inactivate the microorganisms and bacteria within the chamber 36.

In one example, the heater 26 is responsive to instructions from the control unit 24 such that the chamber 36 reaches a temperature of about 190° F. (about 88° C.) for about 3 minutes. In another example, the period of time is about 5 minutes. In other examples, the temperature is above about 140° F., which is a temperature above which most bacteria are killed. Such a combination of heat, time, and exposure to UV light disinfects the CPAP components 22 without damaging the same. In one example, about 99% of all microorganisms and bacteria are killed or inactivated through use of the device 20. In other examples, however, the device 20 does not include the heater 26 or the blower 28, and instead the control unit 24 activates the UV light 30 for the period of time. Without the heater 26 or the blower 28, the period of time may be longer than 3 minutes, such as about 5 minutes.

While first and second dials 42, 44 are shown, in another example, the control unit 24 is pre-programmed to operate disinfection cycle. In one particular example, the control unit 24 is pre-programmed to operate the heater 26, blower 28, and UV light 30 for a predefined period of time and at predefined levels. In this alternate example, a patient would simply close the lid 32 and depress an “on” button, if present, at which point the control unit 24 to begin the pre-defined disinfecting cycle.

FIGS. 3 and 4 illustrate an example device 120 configured to disinfect CPAP components 122 (FIG. 4). To the extent not otherwise described or shown, the device 120 in FIGS. 3 and 4 corresponds to the device 20 of FIGS. 1 and 2, with like parts having reference numerals preappended with a “1.”

Whereas the device 20 of FIGS. 1 and 2 may be particularly useful in a laboratory setting, the device 120 may be particularly useful in residential applications. In particular, the device 120 includes a control unit 124 that is pre-programmed to run a disinfection cycle when a user depresses an “on” button, and when the chamber 136 is closed. The control unit 124 is pre-programmed to run for a set time, which is a predefined period of time. In one example, the predefined period of time is about 5 minutes. The device 120 does not require the user to set any operating parameters of the disinfection cycle, and instead merely requires the user to insert their CPAP components 122 (FIG. 4) into the chamber 136, close the chamber 136, and press the “on” button.

The device 120 includes a base 134 and a drawer 150 slidably mounted to the base 134 and moveable relative to the base 134 between an open position (FIG. 4) and a closed position (FIG. 3). The base 134 and drawer 150 provide boundaries of the chamber 136 when the drawer 150 is in the closed position. The control unit 124 is configured to determine when the drawer 150 is closed, such by interpreting a signal from a sensor. One example sensor is a magnetic switch 152. The magnetic switch 152 may also serve to hold the drawer 150 in the closed position.

In one example, in order to increase the safety of the device 120, the control unit 124 only activates the UV light 130 when the drawer 150 is closed. If a user opens the drawer 150 mid-cycle, for example, the control unit 124 is configured to turn off the UV light 130.

Unlike the device 20, the device 120 does not include a heater or a blower. Rather, disinfection is performed solely by the UV light 130. In order to increase the reach of the UV light 130, the interior of the drawer 150 may be lined with a reflective material, such as polished aluminum. In this way, the UV light emitted by the UV light 130 is reflected within the chamber 136, which increases the surface area of the components 122 exposed to UV light.

As mentioned, the device 120 is configured to run a predefined disinfection cycle. To this end, the device 120 includes a button 154, which is an “on”/“off” button, a first status light 156, and a second status light 158. The first and second status lights 156, 158 may be replaced by a single status light in some examples. The button 154 and the status lights 156, 158 are electrically coupled to the control unit 124.

In one example disinfection cycle, a user opens the drawer 150, as shown in FIG. 4. The user then inserts one or more CPAP components 122 into the chamber 136. The user then shuts the drawer 150, as shown in FIG. 3, and depresses the button 154 to turn on the disinfection cycle. The control unit 124 is configured to determine that the drawer 150 is closed by way of a signal from the magnetic switch 152. When the drawer 150 is confirmed closed, the control unit 124 activates the UV light 130, which then emits UV light, and in particular emits UV-C light, such as that discussed above, for a predefined period of time. As above, the predefined period of time is about five (5) minutes. This period of time is set in a factory setting and stored on the control unit 124. In this example, the user is not allowed to change the predefined period of time.

During the disinfection cycle, the control unit 124 is configured to illuminate the first status light 156. The first status light 156 may be a red light, which indicates that the cycle is ongoing and should not be interrupted. After the predefined period of time, the control unit 124 deactivates the UV light 130, deactivates the first status light 156, and activates the second status light 158, which may be a green light indicating that the disinfection cycle is complete. Optionally, the base 134 may include a tinted lens 160 to allow a user to see into the chamber 136 during the disinfection cycle, while filtering the UV light.

The devices 20, 120 provide a reliable, quick, and relatively easy solution for cleaning and disinfecting CPAP components. The ease of use of the devices 20, 120, coupled with the relatively fast process, relieves a burden on patients faced with cleaning CPAP components on a regular basis. The devices 20, 120 are also less expensive than other higher cost systems on the market, such as those including air purifiers and ozone (which can be a lung irritant), those including ethylene oxide (which is dangerous), and those including chemical bactericides.

It should be understood that terms such as “generally,” “substantially,” and “about” are not intended to be boundaryless terms, and should be interpreted consistent with the way one skilled in the art would interpret those terms.

Although the different examples have the specific components shown in the illustrations, embodiments of this disclosure are not limited to those particular combinations. It is possible to use some of the components or features from one of the examples in combination with features or components from another one of the examples.

One of ordinary skill in this art would understand that the above-described embodiments are exemplary and non-limiting. That is, modifications of this disclosure would come within the scope of the claims. Accordingly, the following claims should be studied to determine their true scope and content.

1. A device for disinfecting components of a continuous positive airway pressure (CPAP) system, comprising:
a chamber;
an ultraviolet (UV) light configured to emit UV light within the chamber; and
a control unit configured to activate the UV light for a period of time.

2. The device as recited in claim 1, wherein the UV light emits UV-C light.

3. The device as recited in claim 2, wherein the UV light emits UV light at a wavelength of 254 nanometers (nm).

4. The device as recited in claim 2, wherein the UV light includes a 13 Watt UV-C bulb.

5. The device as recited in claim 1, further comprising:
a base; and
a drawer slidably mounted to the base and moveable relative to the base between an open position and a closed position, wherein the base and drawer provide boundaries of the chamber when the drawer is in the closed position.

6. The device as recited in claim 5, wherein the control unit is configured such that the UV light is only activated when the drawer is in the closed position.

7. The device as recited in claim 5, wherein the drawer is lined with a reflective material.

8. The device as recited in claim 7, wherein the reflective material is aluminum.

9. The device as recited in claim 1, further comprising:
a base; and
a lid pivotably mounted to the base and moveable relative to the base between an open position and a closed position, wherein the base and lid provide boundaries of the chamber when the lid is in the closed position.

10. The device as recited in claim 9, wherein the control unit is configured such that the UV light is only activated when the lid is in the closed position.

11. The device as recited in claim 9, further comprising:
a heater, the control unit configured to activate the heater and the UV light for the period of time.

12. The device as recited in claim 11, further comprising:
a blower, the control unit configured to activate the blower for the period of time.

13. The device as recited in claim 1, wherein the period of time is a predefined period of time.

14. The device as recited in claim 13, wherein the period of time is about 5 minutes.

15. A method for disinfecting a component of a continuous positive airway pressure (CPAP) system, comprising:
directing ultraviolet (UV) light onto a CPAP component within a chamber for a time period.

16. The method as recited in claim 15, wherein the period of time is about 5 minutes.

17. The method as recited in claim 15, wherein the UV light is UV-C light.

18. The method as recited in claim 15, wherein the UV light is at a wavelength of 254 nanometers (nm).

19. The method as recited in claim 15, wherein a boundary of the chamber is provided in part by one of a lid and a drawer, and the UV light is only directed onto the CPAP component when the one of the lid and the drawer is closed.

20. The method as recited in claim 15, further comprising:
directing heat to the CPAP component for the period of time; and
activating a blower for the period of time.
